(19) United States
(12) Patent Application Publication
(10) Pub. No.: US 2017/0035621 A1
(43) Pub. Date: Feb. 9, 2017

(54) TAMPONS AND METHODS OF FORMING THE SAME

(71) Applicant: FIRST QUALITY HYGIENIC, INC., GREAT NECK, NY (US)

(72) Inventors: Christopher J. Graham, Lock Haven, PA (US); Steven Jeffrey Zimmerman, Howard, PA (US); William M. Child, Lock Haven, PA (US); Babak Damaghi, Kings Point, NY (US)

(21) Appl. No.: 15/094,541

(22) Filed: Apr. 8, 2016

Related U.S. Application Data
(60) Provisional application No. 62/145,564, filed on Apr. 10, 2015.

(51) Int. Cl. A61F 13/20; A61F 13/26

(52) U.S. Cl. CPC A61F 13/2094; A61F 13/2068; A61F 13/26

(57) ABSTRACT

A tampon produced by a method of multiple successive passes of a needle through a fiber web in a manner such that the tampon is afforded a desirable structural configuration and optimized characteristics, including tensile strength, relative absorbency, linear density, and thickness.

### RELATED APPLICATION

This application is a non-provisional based on and claiming priority to U.S. Provisional Patent Application No. 62/145,564, filed Apr. 10, 2015, the contents of which are incorporated herein by reference in their entirety.

### FIELD

The present invention relates to tampons, and in particular, to a tampon manufactured using an optimized needling process.

### SUMMARY

The present invention provides a tampon produced from a nonwoven article that has desirable physical properties that depend on a particular manner in which the nonwoven article is formed.

Accordingly, it is an object of the present invention to provide a tampon that is produced by a method of multiple successive passes of a needle through a fiber web in a manner such that the tampon is afforded a desirable structural configuration. Optimization of the needling process, specifically, varying the number of needle punches during manufacturing, can affect important tampon properties, such as tensile strength, relative absorbency, linear density, and thickness. In particular, these physical properties are dependent upon the particular number and/or frequency with which needles pass or punch through the fiber web to form a tampon. Thus, novel and optimal ranges of these properties have been discovered to form a superior tampon that provides full or near full utilization of absorbent capacity of a carded, needled punched rayon, while still maintaining acceptable fiber integrity.

According to an exemplary embodiment of the present invention, a nonwoven tampon is disclosed, and comprises a body that includes a plurality of fibers extending in a generally lengthwise direction that alternates with a second plurality of fibers extending along a transverse direction. In exemplary embodiments, a needling range of 15.2 to 45.5 punches/cm² may provide the best combination of tensile strength, relative absorbency, linear density, and thickness. In exemplary embodiments, the tampon has a tensile strength between 6.1 lbs and 23 lbs, a relative absorbency between 4.7 g/g and 5.2 g/g, a linear density between 1.8 g/cm and 2.7 g/cm, and a thickness between 6.0 mm and 7.8 mm.

In embodiments, the elongated member has a cylindrical geometry.

In embodiments, the elongated member has a parallelepiped geometry.

In embodiments, the tampon further comprises a withdrawal string extending outward from a proximal end of the elongated member.

In embodiments, the nonwoven web further comprises a second plurality of fibers extending generally perpendicular to the longitudinal axis of the elongated member.

In embodiments, the first plurality of fibers and the second plurality of fibers comprise cellulosic fibers.

In embodiments, the tampon further comprises a non-woven overwrap that covers the elongated member.

In embodiments, a tampon applicator in combination with a tampon is provided. It comprises an elongated member having a longitudinal axis, the elongated member comprising a first plurality of fibers extending generally parallel to the longitudinal axis of the elongated member, an outer tube containing the tampon, and one or more inner tubes telescopically and coaxially mounted within the outer tube so that the inner tube is moveable relative to the outer tube to eject the tampon from the outer tube, the elongated member having a Tensile Strength Factor between 6.1 lbs and 23 lbs, a relative absorbency between 4.7 g/g and 5.2 g/g, a linear density between 1.8 g/cm and 2.7 g/cm, and a thickness between 6.0 mm and 7.8 mm.

In embodiments, the inner tube holds a portion of the tampon with the inner tube and remaining portion of the tampon coaxially and telescopically mounted within the outer tube so that the tampon is held stationary by the outer tube when the user retracts the inner tube but releases the tampon when the user pushes the inner tube. The elongated member having a Tensile Strength Factor between 6.1 lbs and 23 lbs, a relative absorbency between 4.7 g/g and 5.2 g/g, a linear density between 1.8 g/cm and 2.7 g/cm, and a thickness between 6.0 mm and 7.8 mm.

In embodiments, a tampon can be manufactured by method of: providing a fiber web comprised of a plurality of individual fibers generally extending in a first direction, repeatedly needling said fiber web with a frequency of needle punches corresponding to 10.8 to 45.5 needle punches per cm², forming at least one elongated member from the fiber web so as to form a tampon having a Tensile Strength Factor between 6.1 pounds to 23 pounds.

In embodiments, the elongated member has a relative absorbency between 4.7 g/g and 5.2 g/g, a linear density between 1.8 g/cm and 2.7 g/cm, and a thickness between 6.0 mm and 7.8 mm using the tampon manufacturing method.

These and other features of this invention are described in, or are apparent from, the following detailed description of various exemplary embodiments of this invention.

### BRIEF DESCRIPTION OF DRAWINGS

Various exemplary embodiments of this invention will be described in detail, with reference to the following figures, wherein:

FIG. 1 is a side view of a fiber web according to an exemplary embodiment of the present disclosure;

FIG. 2 is a front view of a needle punching machine according to an exemplary embodiment of the present disclosure;

FIG. 3 is a detail view of a needle according to an exemplary embodiment of the present disclosure;

FIG. 4 is a side view of a tampon produced from the fiber web of FIG. 1 according to an exemplary embodiment of the present disclosure;

FIG. 5 is a cross-sectional view of a tampon applicator that includes the tampon produced from the fiber web of FIG. 1 according to an exemplary embodiment of the present disclosure;

FIG. 6 is a graph illustrating the relationship of a tensile pull maximum load strength of a precursor web to a number of needle punches per cm² used to form the precursor web according to an exemplary embodiment of the present disclosure;

FIG. 7 is a graph illustrating the relationship of a relative measure of absorbency of a tampon to a number of needle punches per cm² used to form the tampon according to an exemplary embodiment of the present disclosure;

FIG. 8 is a graph illustrating the relationship of a linear density of a tampon to a number of needle punches per cm² used to form the tampon according to an exemplary embodiment of the present disclosure; and

FIG. 9 is a graph illustrating the relationship of a thickness of a tampon to a number of needle punches per cm² used to form the tampon according to an exemplary embodiment of the present disclosure.

### DETAILED DESCRIPTION

The present invention relates to tampons, and in particular, to a tampon manufactured using an optimized needling process. A tampon is a cylindrical mass of absorbent material, often used as a feminine hygiene product. Tampons are designed for insertion into the vagina during menstruation to absorb menstrual flow. While in the vagina, the tampon acts as a sponge absorbing menses and preventing fluid that exits the cervix from leaking out of the body.

One way of inserting tampons is by using a “push” type applicator. These applicators generally comprise of a pair of telescopically mounted, coaxial hollow tubes. Tampon applicators may be made of plastic or cardboard, and are similar in design to a syringe. The applicator includes two or more tubes, to form an “outer tube,” or barrel, and “inner tube,” or plunger. The applicator contains the tampon and has a smooth external surface to aid insertion. A user pushes the plunger from its distal end to eject the tampon from the barrel into the body cavity. It is also known to provide tampons without applicators. Tampons without applicators, commonly referred to as digital tampons, may have an indentation for the user's fingers to guide the tampon inside the body cavity or may have smooth ends. Tampons are generally provided with an attached string, which extends outside the vagina to assist in removal of the tampon from the vagina. It is also known to provide cordless tampons that are not visible when worn.

Tampons come in a variety of shapes and applicator styles. While the shapes and applicator styles may differ, the absorbency of the tampon is often standardized between manufacturers or within a regulatory jurisdiction. The primary difference between different tampons is in the pad construction and dimensions. The pad construction, in particular, dictates the way the tampon expands when in use. For example, rectangular pad tampons will expand axially, or increase in length, while digital tampons, will expand radially, or increase in diameter.

The tampon itself, also known as the “pledget,” is typically made of cotton, rayon, or blends of rayon and cotton. Rayon is made from cellulose fibers derived from wood pulp and is typically bleached to whiten the fiber. Fiber finishes such as surfactants are also often used in tampon fibers as a process aid or to increase absorbency. The fibers used in a tampon may be processed using nonwoven technology to construct the tampon pad. The nonwoven process provides a means to impart structural integrity to the pad while retaining other desirable characteristics, such as, for example, fiber absorbency, softness, and resilience, to name a few.

In an exemplary method, needle punched nonwovens are created by mechanically orienting and interlocking the fibers of a spun bonded or carded web. This mechanical interlocking is achieved with thousands of reciprocating barbed felting needles repeatedly passing into and out of the web to displace fibers from a generally transverse orientation to a perpendicular orientation.

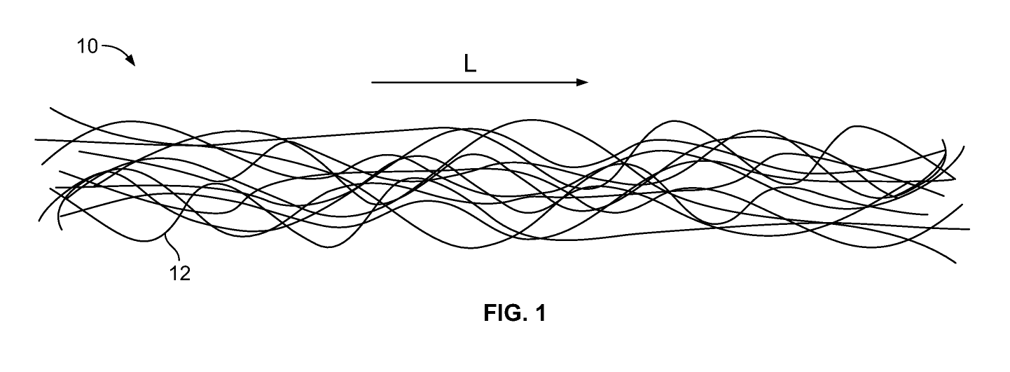

Referring to FIG. 1, a fiber web 10 according to an exemplary embodiment of the present invention is illustrated. Fiber web 10 includes a plurality of fibers 12 that may be strands, lengths, or any other sections or entire lengths of material. Fiber web 10 may be formed from a ribbon, sliver, sheet, or fleece of material that is approximately 3500 mm² in area and may be formed from a single layer or multiple bonded layers of material.

Fiber web 10 may include any number and/or composition of fibers 12, and fibers 12 may have similar and/or varied properties from one another, for example, length, thickness, material composition, cross sectional shape, and/or density, to name a few. In embodiments, fiber web 10 may include fibers 12 formed of one or more materials, for example, polyethylene, polypropylene, polyester, cellulose, rayon, cotton, or other natural materials or blends of polymers and natural materials in any combination or separation. The fibers 12 can also include additives, such as lactic acid to reduce vaginal pH, and any combination of suitable materials. Fibers 12 can be monocomponent, bicomponent and/or biconstituent structures, and may have various configurations such as round and non-round cross-sections and form solid or hollow core tubes (e.g., shaped fibers or capillary channel fibers). In the exemplary embodiment shown, fibers 12 are Galaxy® tri-lobal viscose rayon fibers having a staple length of 30 mm made by Kelheim Fibres of Kelheim Germany.

Formation of the fiber web 10 may be accomplished by re-working bales, piles, or other bunches of unprocessed fibers 12. Such aggregations of fibers 12 may have a substantially non-uniform arrangement so that individual fibers 12 are interlocked in a randomized or otherwise unpatterned fashion. In embodiments, fibers 12 may be picked apart to facilitate later processing steps. A combing unit may be provided to further break apart and align individual fibers 12 such that the individual fibers generally extend along and/or in minimal deviation along a single lengthwise direction L, as shown. In this regard, the fiber web 10 defines a major axis substantially parallel to the lengthwise direction L. As shown, individual fibers 12 may curve, curl, wind and/or alternate as they extend along the lengthwise direction L. The fiber web may be cross-lapped to change the orientation of the fibers and increase web weight per unit area.

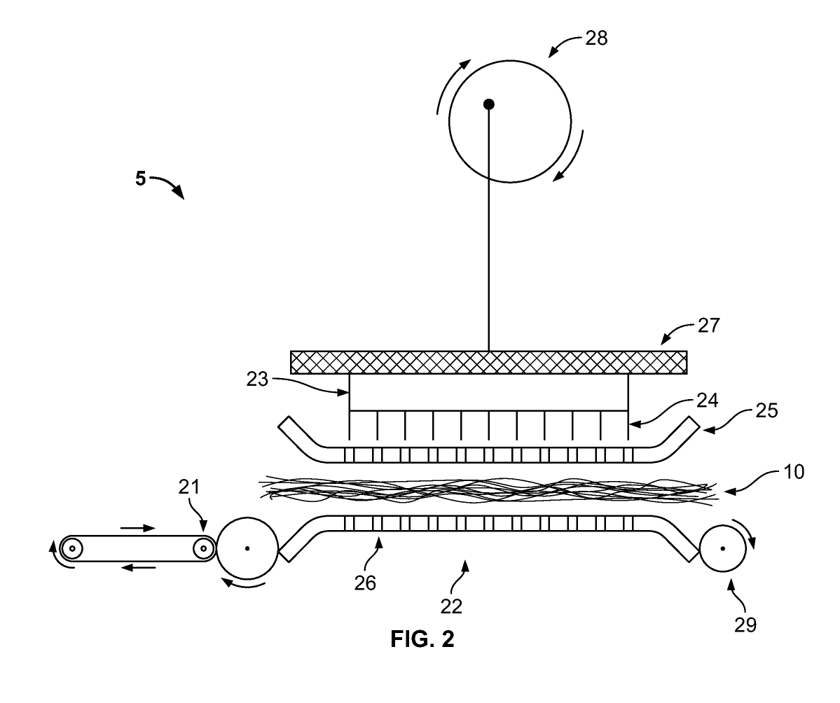

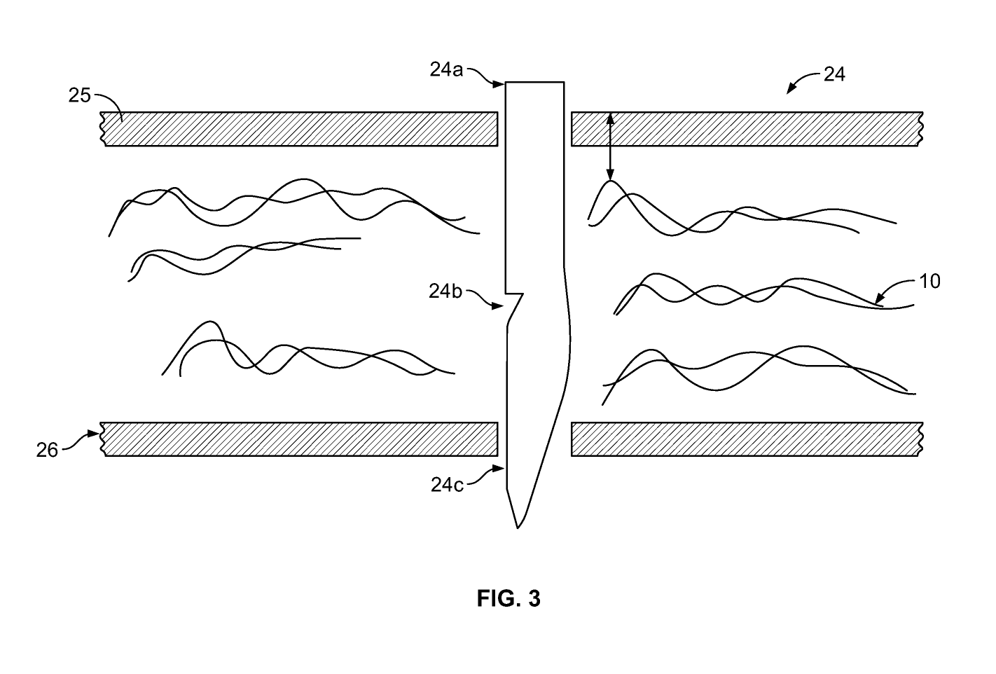

Referring now to FIG. 2 and FIG. 3, a portion of the tampon needle punching manufacturing process and the needle punching machine 5 used therein is shown. A continuous nonwoven fiber web 10 is received from a supply. This fiber web 10 may pass through feed rollers 21 to both slightly compress the web and to draw it into a needling loom 22. The needling loom 22 includes a needle board 23, supporting needles 24, a stripper plate 25, and a bed plate 26. Needles 24 are inserted into and held by the needle board 23. A plurality of needles 24 are provided, usually in the order of several hundred disposed in spaced relation along needle board 23. In exemplary embodiments, needles 24 may be Groz-Beckert felting needles with a 15 gauge shank. In embodiments, needles 24 may have a different configuration.

Needle board 23 is attached to and suspended from needle beam 27, such that the points of needles 24 are facing downward toward fiber web 10 for penetration. Needle beam 27 is attached to a main drive 28 which operates to move the needle board 23 up and down or back and forth. As fiber web 10 passes from feed rollers 21 into needling loom 22, it passes in between stripper plate 25 and bed plate 26. In particular, the fiber web 10 passes under the stripper plate 25 and over the bed plate 26. Stripper plate 25 has perforations positioned so that, upon reciprocation of needle board 23, the needles 24 pass through these perforations and through corresponding perforations in bed plate 26. Needles 24 carry bundles of fibers through the perforations in bed plate 26. Stripper plate 25 strips the fibers from the needles 24 so the material can advance through the needling loom 22.

In exemplary embodiments, each needle 24 includes a shank 24a, one or more barbs 24b, and a blade components 24c. Shank 24a is the thickest part of the needle 24 and fits directly on to needle board 23. As needle beam 27 reciprocates, blades 24c penetrate the fiber batting. Barbs 24b pick up fibers on the downward movement and carry these fibers the depth of penetration. Feed rollers 21 pull the fiber web 10 through needling loom 22 as the needles 24 reorient the fibers from a predominantly horizontal position to an almost vertical position. As the fiber web 10 passes between the stripper plate 25 and bed plate 26, barbs 24b carry and interlock fibers across the thickness of the fiber web 10. The more needles 24 penetrate the fiber web 10, the denser and stronger the fiber web 10 generally becomes. At a certain point, fiber web 10 may be damaged from excessive penetration. The shape and size of the barbs may affect the resulting, needled fiber web 10.

In embodiments, it may be advantageous to perform needling from both sides (i.e., from opposing sides). An example of a commercially available needling device that can needle from both sides is the DI-LOOM OUG-II S35 needle loom. Needling from both sides of fiber web 10 is beneficial because it tends to improve the uniformity of the fiber distribution throughout fiber web 10. In exemplary embodiments, a system offers the capability to vary the number of needles and the fiber web feed rate, so as to provide varying degrees of needle punching in a web. In an exemplary embodiment, the system may have a total of 8994 needles 24, for example, 4497 on the top side and 4497 on the bottom side. Needle board 23 may be fully populated with needles 24, or populated in other configurations, such as, for example, in ⅓ full, half full and ⅔ full configurations, to name a few. In addition, to modulate fiber web's 10 properties, needles may reciprocate at a web feed rate between 4.5 and 8.0 mm/stroke. It should be appreciated to one skilled in the art that various commercially available needling systems, using varying needle patterns and feed rates, may be used to achieve a desired fiber web 10.

The fiber web 10 is then sent through exit rollers 29 on a table or other apparatus for further processing and/or other bonding steps. For example, fiber web 10 may be subject to heat activation and subsequent cooling. Upon completion of such post-processing steps, fiber web 10 may be subdivided into separate stripes, such as slivers or ribbons. Such division of the fiber web 10 may include, for example, stretching, perforating, and/or cutting of the fiber web 10. Each strip may thereafter be subject to one or more forming processes to turn fiber web 10 into a finished tampon.

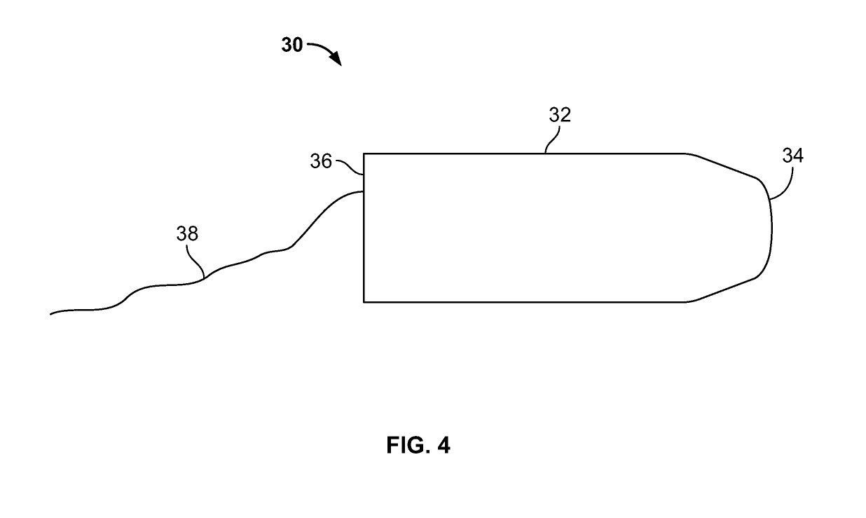

Referring now to FIG. 4, a tampon 30 is shown having been divided from a portion of the fiber web 10 following the needling process described above. Accordingly, the individual fibers 12 of tampon 30, as shown in FIG. 1, together form a body 32 of the tampon 30. As shown, the body 32 of tampon 30 has an elongate configuration and includes an insertion end 34 and a trailing end 36. Insertion end 34 may have a profile that is adapted to dilate and/or move along body tissue, such as, for example, a tapered or contoured profile.

In embodiments, tampon 30 may further include a removal string 38 extending away from the body 32 of tampon 30. Removal string 38 may be attached to tampon 30 in any suitable manner. For example, removal string 38 may be coupled with one or more of fibers 12, as shown in FIG. 1, using sewing, punch and loop or other similar process, during or after formation of tampon 30, and may be disposed in a recess formed within tampon 30. Those skilled in the art will envision other suitable couplings for removal string 38 and tampon 30. In other embodiments, tampon 30 may utilize a nonwoven overwrap, which substantially covers the exterior surface of tampon 30 to help reduce fiber shedding and adhesion to the vaginal mucosa.

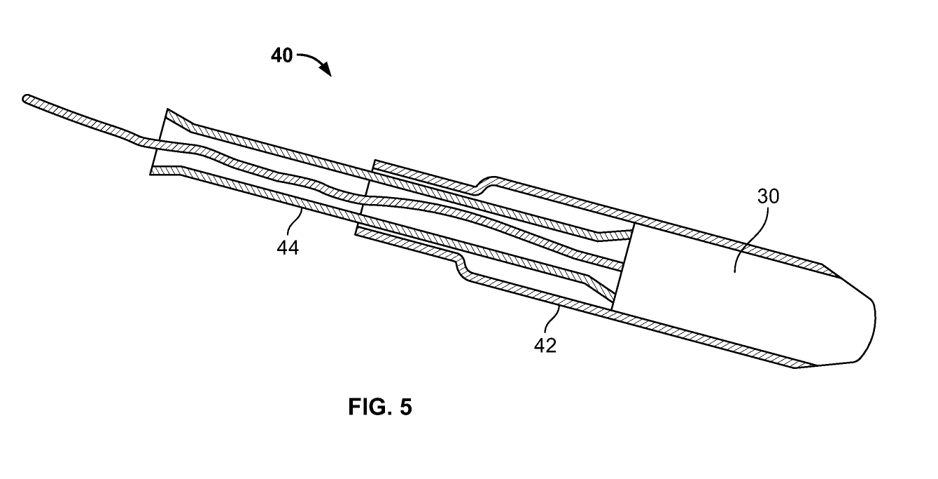

Turning to FIG. 5, a tampon applicator according to an exemplary embodiment of the present invention is generally designated 40, and is shown with tampon 30. Tampon applicator 40 includes a barrel 42 and a plunger 44. As shown, barrel 42 may have a hollow, elongate configuration with a cross-sectional profile similar to that of tampon 30 so that barrel 42 is adapted to at least partially receive the body 32 of tampon 30.

Plunger 44 is insertable into an interior portion of barrel 42, and plunger 44 and barrel 42 are slidably engageable such that a leading portion of the plunger 44 can be advanced into the interior of the barrel 42 to cause the tampon 30 to be pushed from the barrel 42 into the vagina. Since tampon 30 is produced from a section of fiber web 10, tampon has a substantially similar material composition to fiber web 10, and is afforded particular physical properties by virtue of its structural configuration via the altered arrangement, e.g., the arrangement of one or more fibers 12 through the needling process.

It has been discovered that optimization of the needling process, specifically, varying the number of needle punches during manufacturing, can affect important tampon properties, such as tensile pull maximum load strength, relative absorbency, linear density, and thickness. In particular, these physical properties are dependent upon the particular number and/or frequency with which needles 24 pass or punch through fiber web 10 to form tampon 30. Thus, novel and optimal ranges of these properties have been discovered to form a superior tampon that provides full or near full utilization of absorbent capacity of the tampon while still maintaining acceptable fiber integrity.

In this regard, the various physical properties described herein can be measured for a tampon 30 produced from fiber web 10 by the needling process described above at varying needle punch counts per unit area. Such data can be used to predict and plot the relative relationship between properties of a tampon and the number of needle punches per unit area used in the process that forms the tampon 30. Further, such data illuminates optimal ranges of specific properties for a superior tampon, as described further below. In exemplary embodiments, a needling range of 15.2 to 45.5 punches/cm² may provide the best combination of tensile strength, relative absorbency, linear density, and thickness.

As used herein, the Tensile Strength Factor of a tampon made from a precursor web corresponds directly to the tensile pull maximum load strength of 5.0 cm×15.2 cm fiber strips of the precursor web, measured in pounds of force. The tensile pull maximum load strength of 5.0 cm×15.2 cm fiber strips of several precursor webs was measured at varying numbers of needle punches per cm² as shown in Table 1 below. In exemplary embodiments, the Tensile Strength Factor of a finished tampon may be between 3.5 lbs and 23 lbs. In other embodiments, the optimal range for Tensile Strength Factor of the finished tampon is between 6 lbs and 23 lbs.

TABLE 1

| Needle punches/cm² | Tensile Pull Maximum Load Strength (Pounds) |
|---|---|
| 10.8 | 3.5 |
| 12.7 | 5.6 |
| 15.2 | 6.1 |
| 19.2 | 7.6 |
| 21.6 | 10.1 |
| 25.4 | 11.7 |
| 30.3 | 15.0 |
| 32.4 | 18.5 |
| 38.1 | 20.0 |
| 45.5 | 22.9 |
| 57.6 | 23.7 |

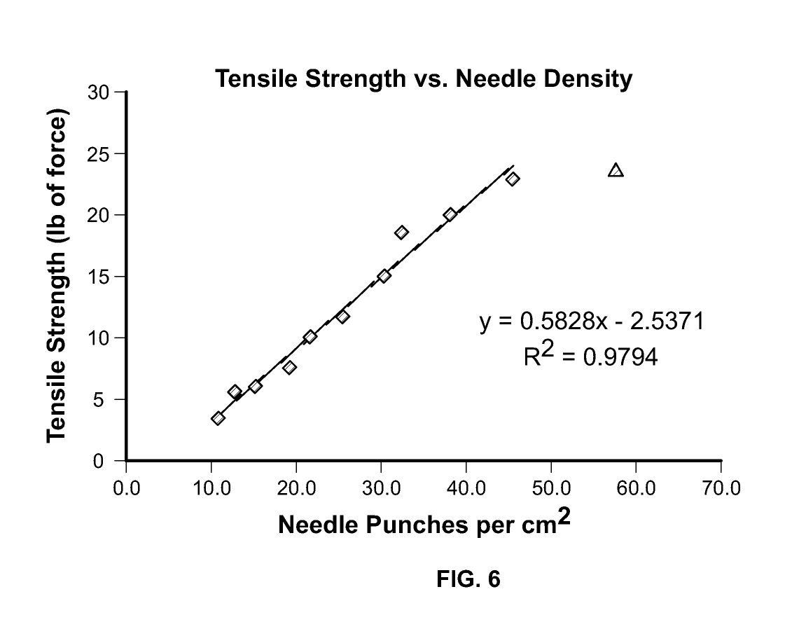

With additional reference to FIG. 6, the Tensile Strength Factor of the tampon described above has a near-linear relationship with respect to the number of needle punches used over an area of a precursor web used to form the tampon on the interval of between about 10.8 and about 45.5 needle punches/cm². Accordingly, the Tensile Strength Factor of a tampon within a designated range can be predicted as a function of the number of needling punches in the following manner:

Tensile Strength Factor (Pounds)=0.58×(punches per cm²)−2.54, with a Pearson's correlation coefficient of ˜0.99.

In an exemplary embodiment, the Syngyna absorbency of a tampon was measured at varying numbers of needle punches as shown in Table 2 below. In the tampon industry, the FDA testing method (21 C.F.R. 801.430) known as the Syngyna test is used to measure absorbency. The Syngyna method comprises a tubular rubber sheath fixed inside a glass jacket to form an artificial vagina. The sheath is set at an angle of about 30 degrees to horizontal and the tampons are positioned inside the membrane with the tampon's center of gravity at the center of the chamber and the withdrawal string extending outside of the opening. Hydrostatic pressure is then applied on the outside of the sheath which collapses around the tampon. Colored test fluid is then admitted to the top of the tampon by a hypodermic needle at a rate slow enough to prevent puddling. When fluid drips from the lower end of the sheath, it is assumed that the tampon is saturated and the absorbent capacity is reached. At this point, the tampon is removed, and the total weight of the test fluid remaining in the tampon is determined. Syngyna absorbency can then be converted to relative absorbency by dividing the mass of fluid absorbed by the mass of the tampon. In exemplary embodiments, the relative absorbency of a finished tampon may be between 4.0 g/g and 5.3 g/g. In other embodiments, the optimal range for relative absorbency of the finished tampon is between 4.7 g/g and 5.2 g/g.

TABLE 2

| Needle punches/cm² | Relative Absorbency (g/g) |
|---|---|
| 10.8 | 5.3 |
| 12.7 | 5.2 |
| 15.2 | 5.2 |
| 19.2 | 5.3 |
| 21.6 | 5.0 |
| 25.4 | 5.1 |
| 30.3 | 4.9 |
| 32.4 | 4.7 |
| 38.1 | 4.7 |
| 45.5 | 4.7 |
| 57.6 | 4.4 |

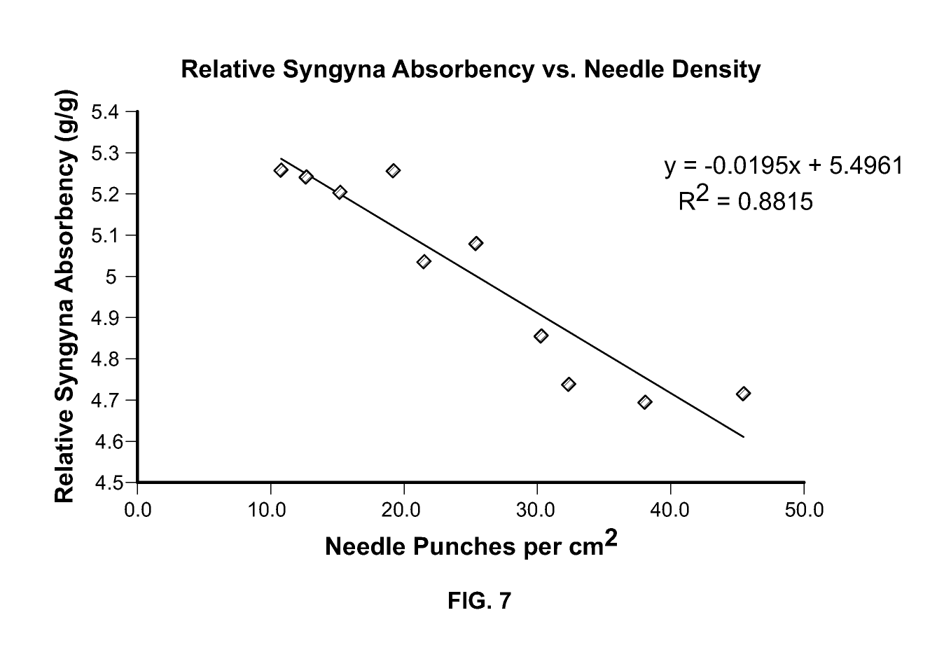

With reference to FIG. 7, the relative absorbency of the tampon described above has a near-linear relationship with respect to the number of needle punches used in forming the tampon on the interval between about 10.8 and 57.6 needle punches per cm². Accordingly, the relative absorbency of a tampon within a designated range can be predicted as a function of the number of needle punches in the following manner:

Relative absorbency (g/g)=−0.020×(punches per cm²)+5.50, with a Pearson's correlation coefficient of about −0.94.

In an exemplary embodiment, the linear density or weight per thickness (measured in grams/cm), of a tampon was measured at varying numbers of needle punches of the portion of a fiber web used to produce the tampon as shown in Table 3 below. For the thickness measurement, a Testing Machines International (TMI) micrometer with a 2 inch presser foot may be used with 95 g/ in² of force applied. Weight was measured on a standard laboratory balance. In exemplary embodiments, the linear density of a finished tampon may be between 1.9g/cm and 2.7 g/cm. In other embodiments, the optimal range for linear density of the finished tampon is between 1.8 g/cm and 2.7g/cm.

TABLE 3

| Needle punches/cm² | Linear Density (g/cm) |
|---|---|
| 10.8 | 1.9 |
| 12.7 | 1.9 |
| 15.2 | 1.8 |
| 19.2 | 1.9 |
| 21.6 | 2.1 |
| 25.4 | 2.2 |
| 30.3 | 2.3 |
| 32.4 | 2.5 |
| 38.1 | 2.6 |
| 45.5 | 2.7 |
| 57.6 | 2.8 |

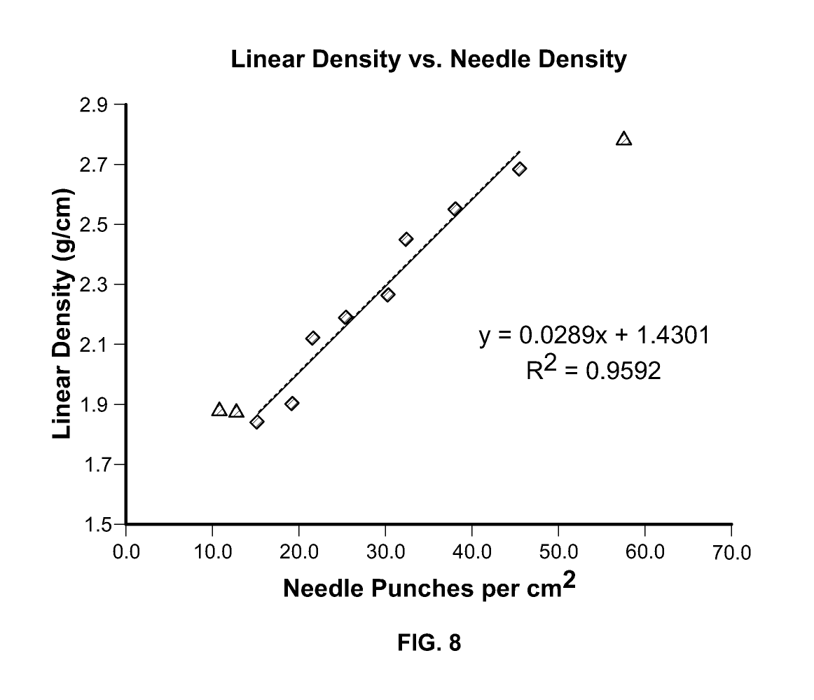

With reference to FIG. 8, the linear density (measured in g/cm) of a tampon has a near-linear relationship with respect to the number of needle punches per cm² of the fiber web used in forming the tampon, for example, between about 15.2 needle punches/cm² and about 45.5 needle punches/cm². Accordingly, the linear density of a tampon within a designated range can be expressed as a function of the number of needle punches in the following manner:

Linear density (g/cm)=0.029×(needle punches per cm²)+1.43, with a Pearson's correlation coefficient of ˜0.98.

In an exemplary embodiment, the thickness (measured in mm) of a tampon was measured, using the aforementioned TMI micrometer method, at varying densities of needle punches per cm² used in forming the tampon as shown in Table 4 below. In exemplary embodiments, the thickness of a finished tampon may be between 6.0 mm and 7.8 mm. In other embodiments, the optimal range for thickness of the finished tampon is between 6.5 mm and 7.5 mm.

TABLE 4

| Needle punches/cm² | Thickness (mm) |
|---|---|
| 10.8 | 7.4 |
| 12.7 | 7.6 |
| 15.2 | 7.8 |
| 19.2 | 7.6 |
| 21.6 | 7.1 |
| 25.4 | 7.0 |
| 30.3 | 7.0 |
| 32.4 | 6.5 |
| 38.1 | 6.3 |
| 45.5 | 6.0 |
| 57.6 | 6.0 |

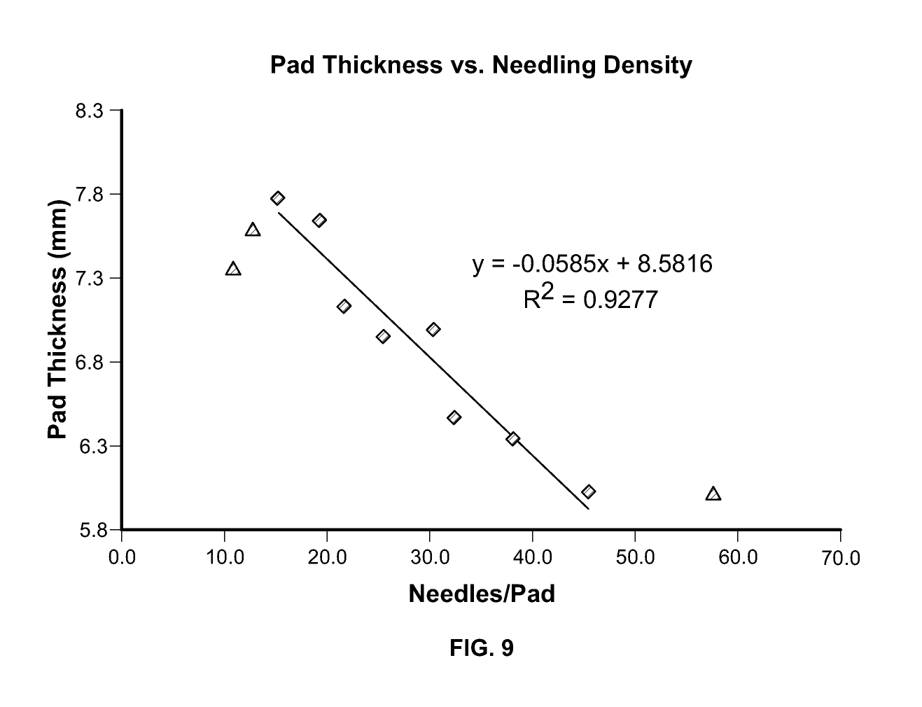

With reference to FIG. 9, the thickness of a tampon has a near-linear relationship with respect to needle punches per cm² used in forming the tampon from a fiber web, for example, between about 15.2 needle punches/cm² and about 45.5 needle punches/cm². Accordingly, the thickness of a tampon within a designated range can be expressed as a function of density number of needle punches in the following manner:

Thickness (mm)=−0.0585×(# of needle punches per cm²)+8.58, with Pearson's correlation coefficient of about −0.96.

In this regard, a tampon may be produced by using the equations above to achieve a desirable combination of physical properties. For example, since each of the properties of tensile pull maximum strength, relative absorbency, linear density, and thickness of a tampon have been expressed as having a linear dependency upon the number of needle punches per cm² used to form the tampon, these linear equations can be balanced against one another to identify a number of needle punches per cm² used to produce a tampon having a simultaneous desirable set of properties, for example, tensile strength, relative absorbency, linear density, and thickness. In exemplary embodiments, it will be understood that additional and/or alternative properties of a tampon may be identified as having a dependency upon the number of needle punches per cm² used to produce the tampon, and may be utilized separately and/or in combination with other properties to find an optimized number of needle punches per cm² to produce a tampon.

While this invention has been described in conjunction with the embodiments outlined above, it is evident that many alternatives, modifications and variations will be apparent to those skilled in the art. Accordingly, the exemplary embodiments of the invention, as set forth above, are intended to be illustrative, not limiting. Various changes may be made without departing from the spirit and scope of the invention.

What is claimed is:

1. A tampon, comprising:
an elongated member having a longitudinal axis and comprising a nonwoven web consolidated via needle punching, the nonwoven web comprising a first plurality of fibers extending generally parallel to the longitudinal axis of the elongated member, the elongated member having a Tensile Strength Factor of between 6.1 lbs and 23 lbs, a Relative Absorbency between 4.7 and 5.2 g/g, a Linear Density between 1.8 to 2.7 g/cm, and a Thickness between 6.0 and 7.8 mm.

2. The tampon of claim 1, wherein the elongated member has a cylindrical geometry.

3. The tampon of claim 1, wherein the elongated member has a parallelepiped geometry.

4. The tampon of claim 1, further comprising a withdrawal string extending outward from a proximal end of the elongated member.

5. The tampon of claim 1, wherein the nonwoven web further comprises a second plurality of fibers extending generally perpendicular to the longitudinal axis of the elongated member.

6. The tampon of claim 1, wherein the first plurality of fibers and the second plurality of fibers comprise cellulosic fibers.

7. The tampon of claim 1, further comprising a non-woven overwrap that covers the elongated member.

8. A tampon applicator in combination with a tampon, comprising:
a tampon comprising an elongated member having a longitudinal axis, the elongated member comprising a first plurality of fibers extending generally parallel to the longitudinal axis of the elongated member;
an outer tube containing the tampon; and
one or more inner tubes telescopically and coaxially mounted within the outer tube so that the inner tube is moveable relative to the outer tube to eject the tampon from the outer tube;
the elongated member having a Tensile Strength Factor between 6.1 lbs and 23 lbs, a relative absorbency between 4.7 g/g and 5.2 g/g, a linear density between 1.8 g/cm and 2.7 g/cm, and a thickness between 6.0 mm and 7.8 mm.

9. A tampon applicator in combination with a tampon, comprising:
a tampon comprising an elongated member having a longitudinal axis, the elongated member comprising a plurality of fibers extending generally parallel to the longitudinal axis of the elongated member;
an inner tube holding a portion of the tampon with the inner tube and remaining portion of the tampon coaxially and telescopically mounted within the outer tube so that the tampon is held stationary by the outer tube when the user retracts the inner tube but releases the tampon when the user pushes the inner tube;
and the elongated member having a Tensile Strength Factor between 6.1 lbs and 23 lbs, a relative absorbency between 4.7 g/g and 5.2 g/g, a linear density between 1.8 g/cm and 2.7 g/cm, and a thickness between 6.0 mm and 7.8 mm.

10. A method of manufacturing a tampon, comprising:
(a) providing a fiber web comprised of a plurality of individual fibers generally extending in a first direction;
(b) repeatedly needling said fiber web with a frequency of needle punches corresponding to 10.8 to 45.5 needle punches per cm²;
(c) forming at least one elongated member from the fiber web so as to form a tampon having a Tensile Strength Factor between 6.1 pounds to 23 pounds.

11. The method of claim 9 wherein said elongated member has a relative absorbency between 4.7 g/g and 5.2 g/g, a linear density between 1.8 g/cm and 2.7 g/cm, and a thickness between 6.0 mm and 7.8 mm.
